(19) United States
(12) Patent Application Publication
(10) Pub. No.: US 2015/0335693 A1
(43) Pub. Date: Nov. 26, 2015

(54) Anti-Melanoma Activity of Cynanchi Atrati Radix Extracts and Manufacturing Thereof

(71) Applicant: Daejeon University Industry-University Cooperation Foundation, Daejeon (KR)

(72) Inventor: Chang Gue Son, Daejeon (KR)

(21) Appl. No.: 14/717,474

(22) Filed: May 20, 2015

(30) Foreign Application Priority Data
May 20, 2014 (KR) 10-2014-0060388

(51) Int. Cl. A61K 36/27

(52) U.S. Cl. CPC A61K 36/27

(57) ABSTRACT

Provided is a preparation method of an anti-tumor composition having a cancer cell proliferation-suppressing effect containing Cynanchi atrati Radix extracts obtained by solvent-extracting Cynanchi atrati Radix, which is a medicinal herb having the cancer cell proliferation-suppressing effect. The Cynanchi atrati Radix extracts may have a melanoma-specific cancer cell proliferation-suppressing effect, thereby making it possible to provide an anti-tumor composition for preventing and treating malignant melanoma.

### TECHNICAL FIELD

The following disclosure relates to Cynanchi atrati Radix extracts having a cancer cell proliferation-suppressing effect and a preparation method thereof.

### BACKGROUND

Cancer is caused by various carcinogens. The kinds of cancer cells are various depending on the various carcinogens. Among them, malignant melanoma, which is a tumor that occurs due to malignancy of melanin pigment producing melanocytes, may occur anywhere melanocytes are present, but mainly occurs on the skin, and has the highest-malignancy grade among cancers occurring on the skin.

Although incidence rate of malignant melanoma is not high in Korea as compared to the West, the incidence rate is increased with age, such that malignant melanoma is significantly rare in persons under the age of 19, but the incidence rate has slowly increased in persons in their 20s while rapidly increased in persons over the age of 40.

A cause of melanoma is not clearly found, but in a type of melanoma frequently occurring in white people, genetic factors, UV exposure, and the like, have been considered as the most important causes. However, while melanoma that occurs from an original mole has been rarely reported in Korea, a type of melanoma that mainly occurs in Korean (a type of malignant melanoma occurring on distal end sites such as the sole of the foot and the subungual site) is not associated with UV exposure.

Among skin cancers, melanoma most frequently generates metastasis and may spread to any organ such as the lymph nodes, bone, liver, spleen, central nervous system, and the like, in addition to spreading to the nearby skin.

As a treatment method of melanoma, early diagnosis and complete resection of lesions is the basis of treatment. In the case in which melanoma spreads, combination chemotherapy dose not have a large effect, but immunotherapy using interferon alpha may be used, and radiation therapy may be used according to a site of lesion.

Chemical anti-cancer drugs mostly act on cell proliferation to exhibit effects. However, since the chemical anti-cancer drugs do not have selectivity to cancer cells, which cause toxic effects on normal cells, a novel anti-cancer drug with excellent selectivity to cancer cells, low toxicity and capability of overcoming drug-resistance should be developed.

Recently, screening and development of an anti-cancer component from natural product with excellent effects on the human body and low toxicity have been spotlighted. For example, anti-cancer screening was performed mainly on plants up to 1982 by U.S. National Cancer Institute (NCI), and as a result of anti-cancer screening on 110,000 plant extracts from about 30,000 plants, taxol from Tazusbrebifolia and camptothecin from Camtotheca acuminate were found.

The natural products, which are secondary metabolites that are present in a trace amount in organisms such as animals, plants, or the like, and materials derived from organisms, such as cells of organism, tissue culture products, or the like, have been used as drugs to treat human's diseases over a long time. In particular, recently, efforts to separate and identify physiologically active materials that directly or indirectly affect the living body from these natural products have been intensively conducted.

Among them, research about a factor promoting toxicity of cancer cells and inhibiting functions of normal cells has been actively conducted to find novel material for fields of cancer prevention and anti-tumor therapeutic drug development in forms of functional foods and new drug development.

Recently, as the incidence rate of malignant melanoma has gradually increased in Korea, it is important to develop a treatment method of melanoma, but as described above, there is no anti-cancer drug of which effects are verified, and there is only localized immunotherapy, so that research into the treatment method of melanoma should be urgently conducted. The natural product has a high potential as a novel raw material of a drug for preventing and treating cancer, and a drug for preventing and treating cancer using this natural product is being actively developed.

### SUMMARY

An embodiment of the present invention is directed to provide a composition for treating melanoma containing Cynanchi atrati Radix extracts corresponding to herb medicine, using properties that Cynanchi atrati Radix extracts can suppress proliferation of malignant melanoma.

Another embodiment of the present invention is directed to provide an anti-melanoma drug containing the composition.

In one general aspect, there is provided a preparation method of an anti-tumor composition having a cancer cell proliferation-suppressing effect, the preparation method including: preparing a medicinal herb including Cynanchi atrati Radix; and immersing the medicinal herb in a solvent or solvent-extracting the medicinal herb to obtain Cynanchi atrati Radix extracts.

The solvent may be a (C₁-C₅) alcohol.

The Cynanchi atrati Radix extracts may be extracted by mixing the medicinal herb with water, a (C₁-C₅) alcohol, or a mixed solution thereof and immersing the medicinal herb therein at room temperature for 10 to 30 hours.

The anti-tumor composition may contain 10 to 90 wt % of the Cynanchi atrati Radix extracts based on a total wt % of the anti-tumor composition.

The cancer cells may be melanoma cells.

In another general aspect, there is provided an anti-tumor composition having a cancer cell proliferation-suppressing effect, obtained by the preparation method as described above.

The composition may contain the Cynanchi atrati Radix extracts obtained by the preparation method as described above or a pharmaceutically acceptable carrier thereof.

In another general aspect, there is provided an anti-cancer drug containing the anti-tumor composition as described above.

### BRIEF DESCRIPTION OF THE DRAWINGS

FIGS. 1A and 1B are graphs illustrating results obtained by measuring a cancer cell proliferation-suppressing effect of Cynanchi atrati Radix extracts according to the present invention.

FIGS. 2A and 2B are views illustrating experimental results obtained by measuring an apoptotic effect of the Cynanchi atrati Radix extracts according to the present invention on B16F10 cells, which are melanoma cells.

FIGS. 3A to 3D are views illustrating results obtained by confirming apoptotic cells in tumor tissue by the Cynanchi atrati Radix extracts according to the present invention.

FIG. 4 is a graph illustrating experimental results obtained by measuring a cell proliferation-suppressing effect of the Cynanchi atrati Radix extracts according to the present invention on CT-26 cells, which are colon cancer cells.

### DETAILED DESCRIPTION OF EMBODIMENTS

Hereinafter, the present invention will be described in detail.

The present inventors has carried out a study and conceived an idea that there is no combination chemotherapeutic agent for malignant melanoma of which effects are verified and among medicinal herbs capable of being obtained from the nature, Cynanchi atrati Radix extracts have a significantly strong melanin production-suppressing effect, and as a result, the present inventors found that the Cynanchi atrati Radix extracts had a cancer cell proliferation-suppressing effect on melanoma, thereby completing the present invention.

Cynanchi atrati Radix, which is a medicinal herb used in the present invention, is a medicine made of a root of Cynanchum atratum Bunge, which a perennial herb belonging to Asclepiadaceae, or congeneric plants thereof, and roots of Cynanachum versicolor Bunge as well as Cynanchi atrati Radix are used in China.

As the effects of Cynanchi atrati Radix, since Cynanchi atrati Radix has a cooling property, Cynanchi atrati Radix may cool heat of the blood meal to treat symptoms such as a slight fever does not appear in the body and the body is tired due to consumption of Qi and blood and fluid and humor in the body at a late stage of warm and hot diseases or after pregnancy, have a significant effect on symptoms such as postpartum false fever, false pyrexia of unknown etiology, and the like, and have an action of reducing fever in the lung to suppress coughing and a diuretic action. Further, Cynanchi atrati Radix is used for fever, coughing caused by lung fever, diuresis, a boil, sore, laryngopharyngitis, and snake bites and does not have toxicity.

The present inventors conceived an idea that Cynanchi atrati Radix, which is a medicinal herb having the above-mentioned effects and being capable of being obtained from the nature, has a significantly strong melanin production-suppressing effect and conducted studies for developing Cynanchi atrati Radix as an anti-tumor composition having an anti-tumor effect against malignant melanoma occupying the largest portion of skin cancers but there is no combination chemotherapeutic agent of which an effect is verified, thereby completing the present invention.

In one general aspect of the present invention, there is provided a preparation method of an anti-tumor composition having a cancer cell proliferation-suppressing effect including: preparing a medicinal herb including Cynanchi atrati Radix; and immersing the medicinal herb in a solvent or solvent-extracting the medicinal herb to obtain Cynanchi atrati Radix extracts.

According to the present invention, the Cynanchi atrati Radix extracts may be obtained by immersing Cynanchi atrati Radix in the solvent or solvent-extracting Cynanchi atrati Radix.

Any kind of Cynanchi atrati Radix may be used as long as it may be purchased as a medicinal herb, and Cynanchi atrati Radix may be used in a grinded powder form, but is not limited thereto.

In another general aspect, the Cynanchi atrati Radix extracts may be extracted by mixing the medicinal herb with water, a (C₁-C₅) alcohol, or a mixed solution thereof and immersing the medicinal herb therein at room temperature for 10 to 30 hours.

In the Cynanchi atrati Radix extracts, the term ‘extracts’ means material obtained by using water, distilled water, the (C₁-C₅) alcohol, or the like, as an extraction solvent. The extraction solvent may be changed depending on the desired anti-cancer activity of the extracts. In the present invention, it is preferable that Cynanchi atrati Radix is extracted using the (C₁-C₅) alcohol, more preferably ethanol.

The (C₁-C₅) alcohol may be used in a form of 100% undiluted solution, but 60 to 90% alcohol may also be used.

As an extraction method of the Cynanchi atrati Radix extracts according to the present invention, any method such as a hot-water extraction method, a heating extraction method, a cold extraction method, an ultrasonic emission method, a stirring method, a mixed method thereof, and the like, may be used.

As the method according to an exemplary embodiment of the present invention, contamination materials are removed by washing Cynanchi atrati Radix purchased as the medicinal herb with water, the Cynanchi atrati Radix is dried and grinded using a grinder, and 60 to 90% ethanol is mixed with the grinded Cynanchi atrati Radix at a weight ratio of 1:1 to 20 using a moving shaker (150 rpm), thereby making it possible to obtain the extracts. The mixing may be performed at room temperature for 10 to 30 hours.

After the mixing is terminated, after only a supernatant is separated and filtered, only a supernatant is separated again using a centrifuge and freeze-dried in a vacuum state, such that the extracts may be obtained, but the present invention is not limited thereto.

As a method according to another exemplary embodiment of the present invention, extracts may be obtained by extracting Cynanchi atrati Radix with methyl alcohol.

The extracts obtained by Cynanchi atrati Radix with methyl alcohol have a high anti-cancer activity.

As a method according to an exemplary embodiment of the present invention, after Cynanchi atrati Radix is washed with distilled water, washed again using an ultrasonic cleaner, dried, grinded to thereby be formed in a powder form, and then, the grinded Cynanchi atrati Radix is immersed in methyl alcohol for 10 to 30 days to thereby be extracted. After petroleum ether is mixed with extracts obtained by filtering and performing centrifugation on a supernatant (hereinafter, methanol extract of Amomum xanthoides (MAX)) obtained by immersion and extraction at a ratio of 1:1 to 20 wt %, the extracts may be separated into an ether layer and a methyl alcohol layer. The extracts in the separated methyl alcohol layer is filtered and centrifuged again, followed by concentration and drying, thereby finally obtaining Cynanchi atrati Radix extracts using methyl alcohol.

In another aspect of the present invention, there is provided an anti-tumor composition containing 10 to 90 wt % of the Cynanchi atrati Radix extracts based on a total wt % of the anti-tumor composition to thereby have the cancer cell proliferation-suppressing effect.

The anti-tumor composition according to the present invention may contain the Cynanchi atrati Radix extracts at an arbitrary content capable of exhibiting a desired anti-cancer activity depending on a use, a formation, a mixing purpose, or the like. The content of the Cynanchi atrati Radix extracts is not particularly limited, but it is preferable that the content the Cynanchi atrati Radix extracts contained in the anti-tumor composition is in a range of 10 to 90 wt % based on a total weight of the anti-tumor composition.

More preferably, the content may be 10 to 50 wt %. In the case in which the content of the Cynanchi atrati Radix extracts is less than 10%, it may be impossible to appropriately exhibit the effects of the Cynanchi atrati Radix extracts.

In another general aspect of the present invention, there is provided an anti-tumor composition having a cancer cell proliferation-suppressing effect, containing Cynanchi atrati Radix extracts obtained by the preparation method as described above.

The Cynanchi atrati Radix extracts according to the present invention, which are extracts having the cancer cell proliferation-suppressing effect, may target cancer cells derived from one or more cell lines selected from colon cancer, cervical cancer, bladder cancer, ovarian cancer, thyroid cancer, prostate cancer, skin cancer, pancreatic cancer, gastric cancer, liver cancer, breast cancer, and lung cancer. Preferably, the Cynanchi atrati Radix extracts may target skin cancer or colon cancer, and more preferably, the Cynanchi atrati Radix extracts may target malignant melanoma among skin cancers.

More specifically, it was confirmed using B16F10 cells (murine melanoma cells) and CT-26 cells (colon carcinoma cells) that the Cynanchi atrati Radix extracts had more excellent anti-cancer effect on melanoma or colon cancer among various cancers.

According to an exemplary embodiment of the present invention, it was confirmed that when B16F10 cells (murine melanoma cells) and CT-26 cells (colon carcinoma cells) were treated with Cynanchi atrati Radix extracts, the Cynanchi atrati Radix extracts had significantly excellent cytotoxicity.

In another general aspect, there is provided an anti-tumor composition having a cancer cell proliferation-suppressing effect, containing the Cynanchi atrati Radix extracts obtained by the preparation method as described above or a pharmaceutically acceptable carrier thereof.

Examples of the pharmaceutically acceptable carrier may include lactose, glucose, sucrose, starch (for example, corn starch, potato starch, and the like), cellulose or a derivative thereof (for example, sodium carboxymethyl cellulose, ethyl cellulose, and the like), malt, gelatin, talc, a solid lubricant (for example, stearic acid, magnesium stearate, and the like), calcium sulfate, vegetable oils (for example, peanut oil, cotton seed oil, sesame oil, olive oil, and the like), polyols (for example, propylene glycol, glycerin, and the like), aliginic acid, emulsifiers (for example, Tween), wetting agents, coloring agents, flavorants, purifying agents, stabilizers, anti-oxidants, preservatives, water, normal saline, phosphate buffer solutions, and the like, but are not limited thereto. One or more carriers may be suitably selected from these carriers as described above depending on a formulation of a pharmaceutical composition according to the present invention.

The anti-tumor composition according to the present invention may be orally or parenterally administered. An administration route of the anti-tumor composition according to the present invention is not particularly limited, but, for example, oral, intravenous, intramuscular, intraarterial, intramedullary, intradural, intracardiac, transdermal, subcutaneous, abdominal, enteral, sublingual, or local administration may be performed. The anti-tumor composition according to the present invention may be mixed with the pharmaceutically acceptable carrier to thereby be formulated into a suitable formulation for clinical administration.

Further, in the case in which the anti-tumor composition is parenterally, for example, intravenously, intracavernosally, intramuscularly, subcutaneously, and intratubularly injected, it is most preferable that the anti-tumor composition is used in a sterile aqueous solution form. In this case, the solution may contain other additive so as to be isotonic with blood.

As the additive, a stabilizer, a diluent, and an emulsifier may be further contained.

The stabilizer may be para-hydroxybenzoic acid esters, for example, methyl paraben or propyl paraben; alcohols, for example, chlorobutanol, benzyl alcohol, or phenyl ethyl alcohol; benzalkonium chloride; phenols, for example, phenol or cresol; thimerosal; dehydroacetic acid; sorbic acid; and a mixture thereof.

The diluent may be lactose, mannitol, glucose, sucrose, calcium sulfate, calcium phosphate, hydroxypropyl cellulose, microcrystalline cellulose, water, ethanol, polyethylene glycol, propylene glycol, glycerol, starch, polyvinylpyrrolidone, magnesium metasilicate aluminate, and a mixture thereof.

The emulsifier may be colloidal clay, for example, bentonite or bee gum; metal hydroxide, for example, magnesium hydroxide or aluminum hydroxide; an anionic surfactant, for example, sodium lauryl sulfate or calcium stearate; a cathionic surfactant, for example, benzalkonium chloride; and a non-ionic surfactant, for example, polyoxyethylene alkyl ether, polyoxyethylene sorbitan fatty acid ester, or sucrose fatty acid ester; and a mixture thereof.

In another general aspect of the present invention, there is provided an anti-cancer drug containing the anti-tumor composition according to the present invention.

An effective dose of the anti-tumor composition according to the present invention may be various depending on the weight, the age, the gender, the health status, and the diet of the patient, the administration time, the administration method, the excretion rate, the severity of the disease, or the like, and is not limited. However, a daily dose of the anti-tumor composition may be 0.001 to 200 mg/kg.

Hereinafter, the present invention will be described in detail through Examples and Comparative Examples. These Examples are illustratively provided only for assisting in the understanding, but the present invention is not limited thereto.

### Example 1

### Preparation of Cynanchi Atrati Radix Extracts

1-1. Preparation Method of Cynanchi Atrati Radix Extracts Using Alcohol

After 100 g of Cynanchi atrati Radix purchased (from Daejeon Oriental Medicine Hospital (Daejeon, Republic Korea)) as a medicinal herb was grinded using a grinder, 70% ethanol (J T Baker, PA Center Valley, USA) was mixed with the grinded Cynanchi atrati Radix at a weight ratio of 1:1 using a moving shaker (150 rpm) at room temperature for 24 hours. After the mixing was completed, only a supernatant was separated, filtered using a filter paper (Advantec, Dublin, Calif., US), centrifuged using a centrifuge at 4° C. and 500 rpm for 30 minutes, thereby separating only a soluble fraction. Only the separated fraction was freeze-dried in a vacuum state, thereby finally obtaining Cynanchi atrati Radix extracts. For quality control of the extracts, fingerprints of the main components in the extracts were confirmed by HPLC using reference components.

1-2. Preparation Method of Cynanchi Atrati Radix Extracts Using Methyl Alcohol

1000 g of Cynanchi atrati Radix was washed with distilled water and washed again using an ultrasonic cleaner. Then, the washed Cynanchi atrati Radix was dried in a dry oven at 60° C. A total weight of the Cynanchi atrati Radix prepared in a powder form by grinding the dried Cynanchi atrati Radix using a grinder was 300 g. The Cynanchi atrati Radix prepared in a powder form was immersed in 500 ml of methyl alcohol (J T Baker, PA Center Valley, USA) for 10 days. Only a supernatant (hereinafter, methanol extract of Amomum xanthoides (MAX)) of primary extracts obtained by immersion and extraction was separated, filtered, and centrifuged (150 rpm, 30 minutes), thereby obtaining secondary extracts. Petroleum ether (J T Baker, PA Center Valley, USA) was mixed with the obtained secondary extracts at a weight ratio of 1:1, and then the extracts may be separated into an ether layer and a methyl alcohol layer. After extracts of the separated methyl alcohol layer were filtered and centrifuged (500 rpm, 30 minutes) again, the centrifuged extracts were freeze-dried in a vacuum state, thereby finally obtaining Cynanchi atrati Radix extract using methyl alcohol. Dried extracts (referred to as ‘methanol soluble fraction of Amomum xanthoides (MFAX)) having a weight corresponding to about 6.62% of a weight of initially dried extracts in the powder form were recovered. For quality control of the extracts, fingerprints of the main components in the extracts were confirmed by HPLC using reference components.

### Example 2

### Cell Culture

Cells used in cell culture were B16F10 cell line (murine melanoma cells) purchased from Korean Cell Line Bank and CT-26 cell line (colon carcinoma cells) purchased from ATCC. The B16F10 cell lines and CT26 cell line were cultured in Dulbecco's modified eagle medium (DMEM) containing 10% fetal bovine serum (FBS) and antibiotics (100 U/mL penicillin G, 100 ug/mL streptomycin) at 37° C. under 5% CO₂ conditions.

The cells were cultured in a disposable flask for cell culture, and at the time of an experiment, the cells were cultured in a 96-well plate or 150 mm culture dish according to an experimental method and then used.

### Example 3

### Effect of Cynanchi Atrati Radix Extracts on Cancer Cell

### Example 3-1

### Effect of Cynanchi Atrati Radix Extracts on Melanoma B16F10 Cell

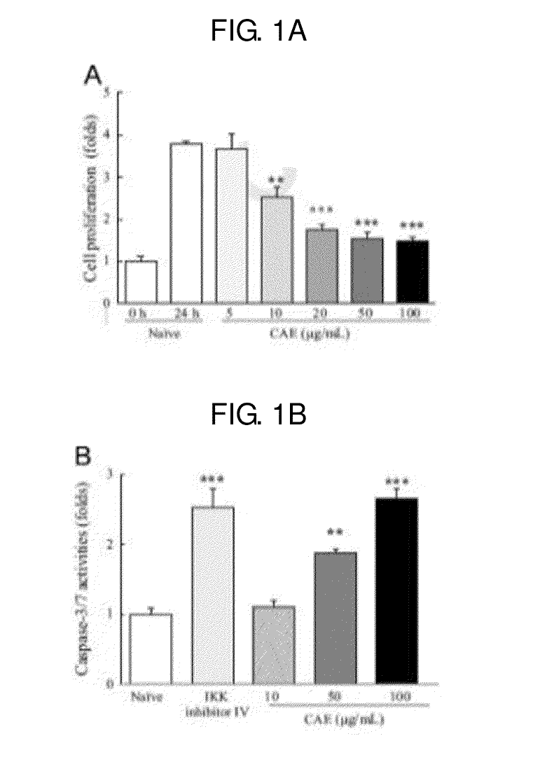

An experiment for measuring a cancer cell proliferation-suppressing effect of the Cynanchi atrati Radix extracts extracted in Example 1 has been conducted. The results were shown in FIGS. 1A and 1B.

The B16F10 cells were seeded in a 96-well plate at 2×10³ cell/well, and then treated with various concentrations (0, 5, 10, 20, 50, 100 ug/mL) of Cynanchi atrati Radix extracts. The cells treated with the Cynanchi atrati Radix extracts were cultured for 24 hours, and then treated with 10% WST-8 reagent (Sigma-Aldrich), followed by performing a reaction for 90 minutes. After the reaction was terminated, absorbance of a supernatant sample (150 ul total volume) of the well was measured at a wavelength of 450 to 600 nm using Soft Max Microplate Reader (Molecular Devices, Sunnyvale, Calif., USA).

As a result of confirming a cancer cell proliferation-suppressing effect of the Cynanchi atrati Radix extracts according to the present invention using the B16F10 cells, among groups treated by various concentrations of the Cynanchi atrati Radix extracts, in a group treated with 10 ug/mL of the Cynanchi atrati Radix extracts (CAE), 50% Inhibitory concentration (IC₅₀) was 11.06 ug/mL, and a B16F10 cell proliferation-suppressing effect was the highest.

### Example 3-2

### Caspase-3/7 Activity of Melanoma B16F10 Cell on Cynanchi Atrati Radix Extracts

An experiment for measuring a change in caspase-3/7 activity caused by an apoptotic effect in the case of treating the B16F10 cells with the CAE extracted in Example 1 was conducted. The results were shown in FIGS. 1A and 1B. At the time of comparing groups treated with various concentrations of the CAE extracted in Example 1 with a group that was not treated with the CAE, in a group treated with 100 ug/mL of the CAE, a caspase-3/7 activity was the highest. As a result, the CAE had the highest effect on apoptosis of the B16F10 (melanoma) cell at the above-mentioned concentration of the CAE.

### Example 3-3

### Effect of Cynanchi Atrati Radix Extracts on CT26 Cell (Colon Cancer Cell)

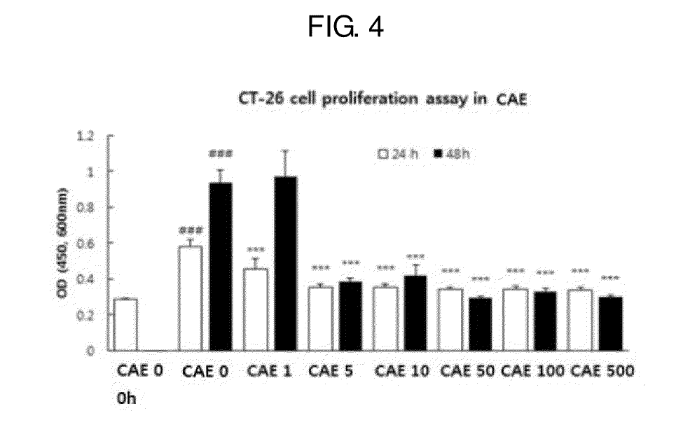

An experiment for measuring a cancer cell proliferation-suppressing effect of the Cynanchi atrati Radix extracts extracted in Example 1 on the CT26 cells (colon cancer cells) has been conducted. The results were shown in FIG. 4.

The CT 26 cells were seeded in a 96-well plate at 4×10³ cell/well, and then treated with various concentrations (0, 1, 5, 10, 50, 100, 200 ug/mL) of Cynanchi atrati Radix extracts. The cells treated with the Cynanchi atrati Radix extracts was cultured for 24 hours and 48 hours, and then treated with 10% WST-8 reagent (Sigma-Aldrich), followed by performing a reaction for 90 minutes. After the reaction was terminated, absorbance of a supernatant sample (150 ul total volume) of the well was measured at a wavelength of 450 to 600 nm using Soft Max Microplate Reader (Molecular Devices, Sunnyvale, Calif., USA).

As a result of confirming a cancer cell proliferation-suppressing effect of the Cynanchi atrati Radix extracts according to the present invention using the CT26 cells, among groups treated with various concentrations of the Cynanchi atrati Radix extracts, in groups treated with 5, 10, 50, 100, and 200 ug/mL of the Cynanchi atrati Radix extracts, a CT26 cell proliferation-suppressing effect was high as compared to groups treated with 0 or 1 ug/mL of the Cynanchi atrati Radix extracts.

### Example 4

### Anti-Tumor Effect of Cynanchi Atrati Radix Extracts

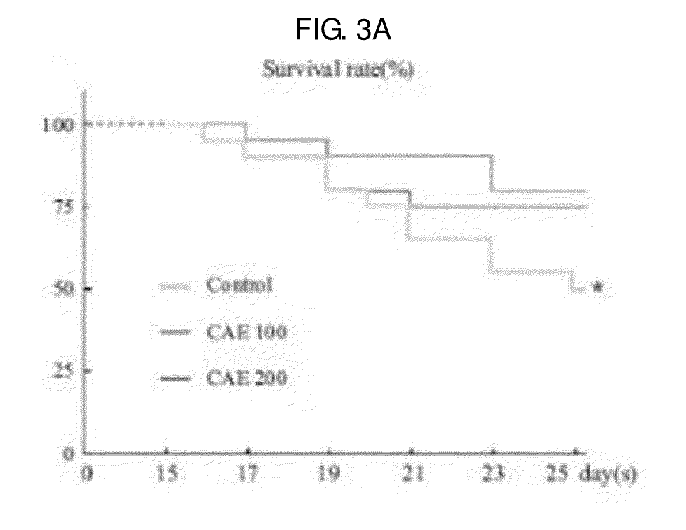

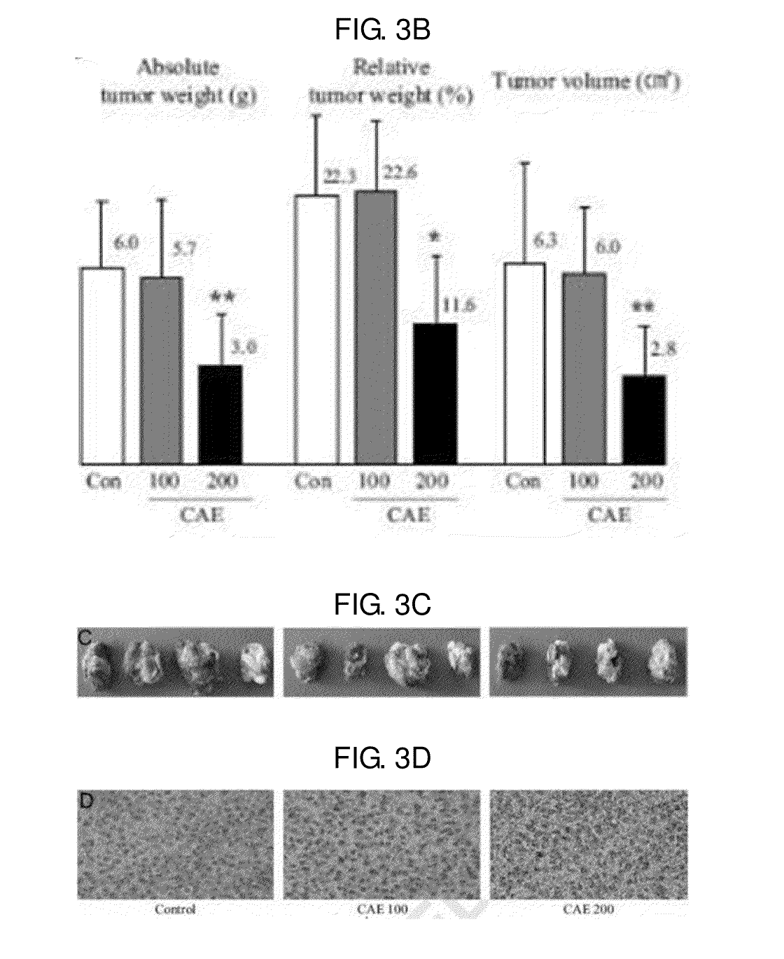

In order to confirm an anti-melanoma effect of the CAE extracted in Example 1, an experiment was conducted using the B16F10 cells. The results were shown in FIGS. 3A to 3D.

In order to examine an anti-melanoma effect, specific-pathogen-free C57BL/6N male mice (Koatech, 12 weeks old, 24-26 g) were purchased and bred under pathogen-free and 12 h light/dark day cycle environments. After injecting the B16F10 cells (1×10⁷ cells) cultured in Example 2 into mice subcutaneous tissue, the mice were monitored for 15 days or more. After 15 days, the mice were classified into three groups (20 mice/group) depending on a size of tumor formed in the mice subcutaneous tissue. The three groups were divided into a control group in which only distilled water was injected into mice with tumor formation, a group in which mice with tumor formation were treated with 100 mg/Kg of the CAE, and a group in which mice with tumor formation were treated with 200 mg/Kg of the CAE, and the mice were treated with the CAE and monitored every day for 10 days. After 10 days, all of the mice were sacrificed, and tumor cells formed in the subcutaneous tissue were extracted in portions as tumor samples for measuring tumor volumes and apoptosis.

As a result of measuring the tumor volumes in three groups, the biggest decrease in the tumor volumes was observed in the group treated with 100 mg/kg of the CAE and the group treated with 200 mg/Kg of the CAE as shown in FIGS. 3A to 3D.

### Example 5

### Measurement of Apoptotic Effect on Cancer Cells

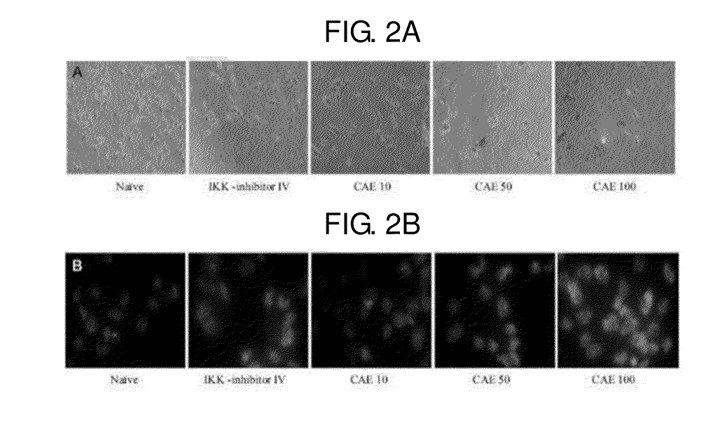

Apoptosis measurement is an experiment for confirming apoptotic cells in the B16F10 cells and tumor tissue and TUNEL assay, which is an experimental method widely used for measuring apoptosis, was performed. The results were shown in FIGS. 2A (apoptosis of B16F10 cell) to 3D (apoptosis in tumor tissue).

In order to confirm apoptotic signal of melanoma cells by the CAE prepared in Example 2, the B16F10 cells cultured in Example 1 were stained with TUNEL and DAPI. The B16F10 cells cultured in Example 1 were treated with each concentration (10, 50, and 100 ug/ml) of the CAE. The apoptotic signal was indicated by a red-brown color, and nuclei of the B16F10 cells had a blue color. As an experimental result, in a group in which the B16F10 cells were treated with 100 ug/mL of the CAE, the strongest apoptotic signal was observed, and at the time of comparing a group that was not treated with groups treated with the CAE, overall, a strong DAPI signal was observed in the groups treated with each concentration of the CAE (FIGS. 2A and 2B). It may be appreciated from the result that an apoptotic response was induced by the CAE.

As a result of confirming apoptosis in the B16F10 cells and tumor tissue by fixing the tumor tissue samples extracted in Example 4 in 10% formalin, preparing tissue samples using paraffin, and performing TUNEL staining thereon, as shown in FIG. 4D, the largest apoptotic changes were observed in groups treated with 100 mg/kg of the CAE and 200 mg/kg of the CAE.

The present invention provides the anti-tumor composition containing Cynanchi atrati Radix extracts having the cancer cell proliferation-suppressing effect.

The anti-tumor composition has an anti-cancer effect specific to malignant melanoma, thereby making it possible to provide an anti-cancer drug for preventing and treating malignant melanoma containing the anti-tumor composition.

What is claimed is:

1. A preparation method of an anti-tumor composition having a cancer cell proliferation-suppressing effect, the preparation method comprising:
preparing a medicinal herb including Cynanchi atrati Radix; and
solvent-extracting the medicinal herb to obtain Cynanchi atrati Radix extracts.

2. The preparation method of claim 1, wherein the solvent is a (C₁-C₅) alcohol.

3. The preparation method of claim 1, wherein the Cynanchi atrati Radix extracts are extracted by mixing the medicinal herb with water, a (C₁-C₅) alcohol, or a mixed solution thereof and immersing or stirring the medicinal herb therein at room temperature for 10 to 30 hours.

4. The preparation method of claim 1, wherein the anti-tumor composition contains 10 to 90 wt % of the Cynanchi atrati Radix extracts based on a total wt % of the anti-tumor composition.

5. The preparation method of claim 4, wherein cancer cells are melanoma cells or colon cancer cells.

6. An anti-tumor composition having a cancer cell proliferation-suppressing effect obtained by the preparation method of claim 1.

7. The anti-tumor composition having a cancer cell proliferation-suppressing effect of claim 6, wherein it comprises the Cynanchi atrati Radix extracts or a pharmaceutically acceptable carrier thereof.

8. An anti-cancer drug comprising the anti-tumor composition of claim 6.

9. An anti-tumor composition having a cancer cell proliferation-suppressing effect obtained by the preparation method of claim 2.

10. An anti-tumor composition having a cancer cell proliferation-suppressing effect obtained by the preparation method of claim 3.

11. An anti-tumor composition having a cancer cell proliferation-suppressing effect obtained by the preparation method of claim 4.

12. An anti-tumor composition having a cancer cell proliferation-suppressing effect obtained by the preparation method of claim 5.
